(19) United States
(12) Patent Application Publication
(10) Pub. No.: US 2016/0145605 A1
(43) Pub. Date: May 26, 2016

(54) PEPTIDE-PRESENTING PROTEIN AND PEPTIDE LIBRARY USING SAME

(71) Applicant: TOKYO INSTITUTE OF TECHNOLOGY, Tokyo (JP)

(72) Inventors: Tetsuya KADONOSONO, Tokyo (JP); Shinae KONDOH, Tokyo (JP)

(73) Assignee: Tokyo Institute of Technology, Tokyo (JP)

(21) Appl. No.: 14/786,416

(22) PCT Filed: Apr. 23, 2014
(86) PCT No.: PCT/JP2014/061366

§ 371 Date: Jan. 12, 2016

(30) Foreign Application Priority Data
Apr. 23, 2013 (JP) 2013-090524

(51) Int. Cl. C12N 15/10; C12N 9/10; C07K 7/08; C07K 7/06; G01N 33/68; C07K 14/435

(52) U.S. Cl. CPC C12N 15/1037; G01N 33/6845; C07K 14/43595; C07K 7/08; C07K 7/06; C12N 9/1088; C07K 2319/60; C12Y 205/01018

(57) ABSTRACT

The object of the present invention is to provide a method for screening peptide(s) highly specific to a target molecule. The object can be solved by a peptide-presenting protein wherein a peptide is presented in a presentation region consisting of the 131st to 139th amino acids in the amino acid sequence of the SEQ ID NO: 54, wherein the peptide consisting of 4 to 15 amino acids is inserted in the 131st to 139th amino acids sequence, or substituted for any of 1 to 9 amino acid(s) sequence in the 131st to 139th amino acids sequence; and a total length of an amino acid sequence corresponding to the 131st to 139th amino acids sequence is 15 or less.

### TECHNICAL FIELD

The present invention relates to a peptide-presenting protein and a peptide library using the same. According to the present invention, peptide(s) highly specific to a target molecule can be screened.

### BACKGROUND ART

Functional peptide(s) having high specificity to a target molecule and binding to a target molecule is useful for a drug-discovery. Thus, in order to obtain the functional peptide(s), many peptide libraries according to various peptide-presenting techniques have been constructed and many high-throughput screening techniques have been developed. As for the methods for presenting the peptide library, display on a phage, E. coli, a yeast, or the like is used. In the phage display, for example, screened peptides are presented as a fusion polypeptide on a bacteriophage surface protein. The peptide-displayed phage particles are brought into contact with a microtiter plate in which the target molecule is immobilized, to thereby conduct an affinity selection. Phages expressing peptide having affinity to target molecule can be concentrated due to this (non-patent literature 1).

In the phage display, the peptide presented on the concentrated phages corresponds one-to-one to a gene cording the same, and therefore, the peptide of interest can be easily identified. Further, the gene can be easily amplified (proliferated), and therefore, the phage display is widely used as the method for screening peptides.

### CITATION LIST

### Non-Patent Literature

[NON-PATENT LITERATURE 1] Science (United States) 1990, Vol. 249, p. 386-390
[NON-PATENT LITERATURE 2] Nature Biotechnology (Great Britain) 2006, Vol. 24, p. 79-88

### SUMMARY OF INVENTION

### Technical Problem

However, peptide(s) obtained by the conventional display method can be bound to the target molecule, but are often bound to other molecules. That is to say, peptide(s) having low specificity to the target molecule may be screened, and thus, the peptide(s) cannot be frequently made fit for practical use as a medicine. Accordingly, the object of the present invention is to provide a method for screening peptide(s) highly specific to a target molecule.

### Solution to Problem

The inventors have conducted intensive studies for the low specificity of the screened peptide(s), and as a result, think that the peptide(s) presented by the conventional display method are screened under the condition that a structure of the peptide is freely changed. Thus, the inventors think that the many peptide(s) obtained as a candidate peptide may also bind to molecules other than the target molecule.

Further, the inventors of the present invention have conducted intensive studies into a method for limiting a flexibility of structure of the presented peptide, and as a result, surprisingly found that peptide(s) capable of specifically binding to the target molecule can be screened by inserting a peptide into the region consisting of the 131st to 139th amino acids of the superfolder GFP (non-patent literature 2; hereinafter sometimes referred to as sfGFP) to limit the flexibility of peptide structure.

The present invention is based on the above findings.

Namely, the present invention relates to:
[1] a peptide-presenting protein wherein a peptide is presented in a presentation region consisting of the 131st to 139th amino acids in the amino acid sequence of the SEQ ID NO: 54, wherein the peptide consisting of four to fifteen amino acids is inserted in the 131st to 139th amino acids sequence, or substituted for any of 1 to 9 amino acid(s) sequence in the 131st to 139th amino acids sequence; and a total length of an amino acid sequence corresponding to the 131st to 139th amino acids sequence is fifteen or less,
[2] the peptide-presenting protein of the item [1], wherein one or plural amino acids are deleted, substituted, inserted, and/or added in the first to 130th amino acids sequence or the 140th to 238th amino acids sequence in the amino acid sequence of SEQ ID NO: 54, and exhibiting a fluorescence generation activity,
[3] the peptide-presenting protein of the item [1] or [2], wherein amino acid sequence(s) of end(s) of the inserted or substituted peptide consisting of four to fifteen amino acids are translated from base sequence(s) comprising a restriction enzyme site,
[4] a fusion protein wherein the peptide-presenting protein of the items [1] to [3], and a surface protein are fused,
[5] a polynucleotide encoding the peptide-presenting protein of the items [1] to [3], or the fusion protein of the item [4],
[6] a vector comprising the polynucleotide of the item [5],
[7] a host comprising the vector of the item [6],
[8] a library comprising the host of the item [7], wherein the presented peptides are a number of peptides consisting of random amino acid sequences,
[9] a protein for presenting a peptide, which can present the peptide in a presentation region consisting of the 131st to 139th amino acids in the amino acid sequence of the SEQ ID NO: 54, wherein the peptide consisting of four to fifteen amino acids is inserted in the 131st to 139th amino acids sequence, or substituted for any of 1 to 9 amino acid(s) sequence in the 131st to 139th amino acids sequence; and a total length of amino acids sequence corresponding to the 131st to 139th amino acids sequence is fifteen or less,
[10] the protein for presenting a peptide of the item [9], wherein one or plural amino acids are deleted, substituted, inserted, and/or added in the first to 130th amino acids sequence in the amino acid sequence or the 140th to 238th amino acids sequence in the amino acid sequence of SEQ ID NO: 54, and exhibiting a fluorescence generation activity,
[11] the protein for presenting a peptide of the item [9] or [10], wherein amino acid sequence(s) of end(s) of the inserted or substituted peptide consisting of four to fifteen amino acids are translated from base sequence(s) comprising a restriction enzyme site,
[12] A fusion protein wherein the protein for presenting a peptide of the items [9] to [10] is fused with a surface protein,
[13] a polynucleotide encoding the protein for presenting a peptide of the items [9] to [11], or the fusion protein of the item [12],
[14] a vector comprising the polynucleotide of the item [13],
[15] a host comprising the vector of the item [14],
[16] a method for constructing a library, comprising the steps of: (1) inserting a nucleotide encoding a peptide having four to 15 amino acids into the vector according to claim 14 to obtain an expression vector, wherein the peptide is inserted in the 131st to 139th amino acids sequence of the presentation region, or substituted for any of 1 to 9 amino acid(s) sequence in the 131st to 139th amino acids sequence of the presentation region; and a total length of amino acids sequence corresponding to the 131st to 139th amino acids sequence is fifteen or less, and (2) introducing the vector into the hosts,
[17] a method for screening a peptide binding to a target molecule, comprising the steps of: (1) bringing into contact the peptide library of the item [8] with the target molecule, (2) recovering hosts binding to the target molecule,
[18] a method for screening a peptide of the item [17], further comprising (3) the step of proliferating the recovered hosts, and
[19] the method for screening a peptide of the item [18], wherein the contact step (1), the host-recovering step (2), and the proliferating step (3) are repeated.

### Advantageous Effects of Invention

According to the peptide-presenting protein and the peptide library using the same of the present invention, the peptide(s) highly specific to a target molecule can be screened. Further, the scaffold protein, i.e. sfGFP, in which the peptide is presented, is a fluorescence protein, and thus, the number of molecules thereof can be easily estimated by fluorescence intensity. Therefore, it is useful for estimating a binding amount of peptide in the screening. The present invention can be applied to the widely used presenting techniques, such as the phage display, E. coli display, yeast display, in vitro virus, and ribosome display. That is, the present invention can be applied to the conventional, high-throughput screening techniques. Furthermore, molecularly-targeted drugs or drug delivery systems can be developed based on the sequence information and structural information of a peptide obtained by the present invention.

### BRIEF DESCRIPTION OF THE DRAWINGS

FIG. 1 is an illustration showing an amino acid sequence of full length sfGFP, and a 131st to 139th amino acids sequence (reversed characters).

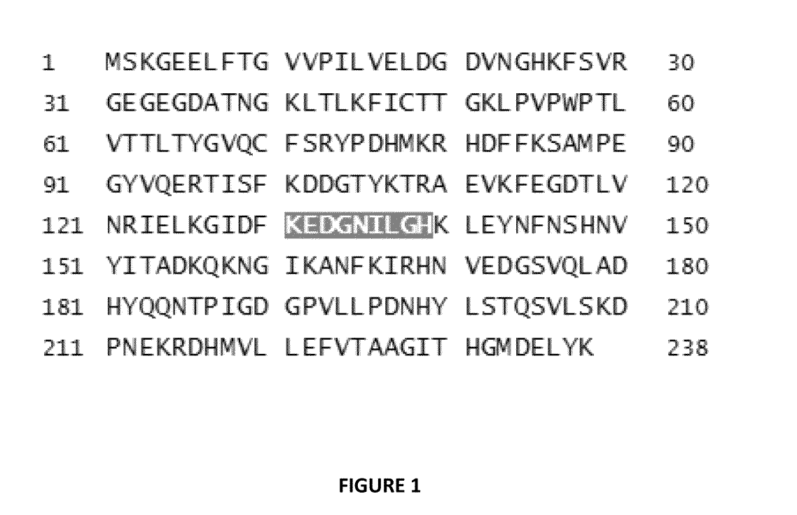

FIG. 2 is a photograph showing that the peptides recognized by caspase 3, which are introduced in Examples 1 to 15 and Comparative Examples 1 to 5, are cleaved by caspase 3.

FIG. 3 is a graph showing fluorescence intensities of peptide-presenting proteins obtained in Examples 1 to 15 and Comparative Examples 1 to 5.

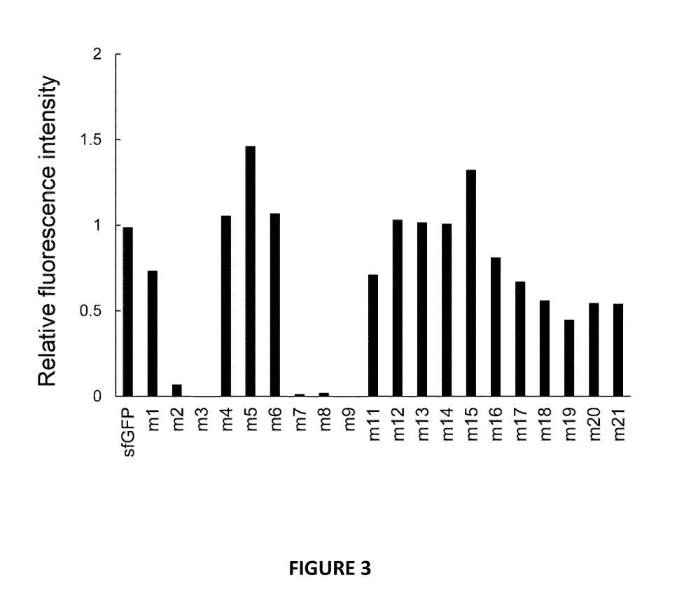

FIG. 4 is illustrations showing molecular dynamics simulations of the peptides (m1 (Example 1), m6 (Example 4), m19 (Example 13), and m7 (Comparative Example 3)) inserted in the peptide-presenting protein.

FIG. 5 is a table showing the peptide sequences capable of binding to a HER2 protein, which is frequently overexpressed in breast cancer cells (A), and photographs showing that the peptide-presenting proteins wherein the above peptides are introduced, bind to HER2.

FIG. 6 is an illustration showing amino acid sequences of the peptide-presenting proteins of the present invention, i.e. m12 (Example 6), m15 (Example 9), m16 (Example 10), m19 (Example 13), m21 (Example 15), and m22 (Example 17) (A), and molecular dynamics simulations thereof (B).

### [1] PEPTIDE-PRESENTING PROTEIN, POLYNUCLEOTIDE, VECTOR, AND HOST

### <<Peptide-Presenting Protein>>

In the peptide-presenting protein of the present invention, a peptide is presented in a presentation region consisting of the 131st to 139th amino acids in the amino acid sequence of the SEQ ID NO: 54, and the peptide consisting of four to fifteen amino acids (preferably 4 to 14 amino acids, more preferably 4 to 13 amino acids, most preferably 4 to 12 amino acids) is inserted in the 131st to 139th amino acids sequence, or substituted for any of 1 to 9 amino acid(s) sequence in the 131st to 139th amino acids sequence; and a total length of amino acids sequence corresponding to the 131st to 139th amino acids sequence is fifteen or less (preferably 14 or less, more preferably 13 or less, most preferably 12 or less). Further, a lower limit of the length of amino acids sequence corresponding to the 131st to 139th amino acids sequence is preferably five or more. The peptide-presenting protein of the present invention may be one wherein one or plural multiple amino acids are deleted, substituted, inserted, and/or added in the first to 130th amino acids sequence or the 140th to 238th amino acids sequence in the amino acid sequence of SEQ ID NO: 54, and exhibiting a fluorescence generation activity. The protein consisting of the amino acid sequence of the SEQ ID NO: 54 is the sfGFP described in Non-patent literature 2. However, GFP-based proteins can be used as the scaffold protein for inserting the peptide in the peptide-presenting protein of the present invention. That is to say, GFP or GFP-based variant protein derived from GFP can be used without limitation. As a concrete scaffold protein, there may be mentioned GFP, YFP, CFP, BFP, or RFP.

Preferably, in the peptide-presenting protein of the present invention, a peptide is presented in a presentation region consisting of the 131st to 138th amino acids in the amino acid sequence of the SEQ ID NO: 54, and the peptide consisting of four to fourteen amino acids (preferably 4 to 13 amino acids, more preferably 4 to 12 amino acids) is inserted in the 131st to 138th amino acids sequence, or substituted for any of 1 to 8 amino acid(s) sequence in the 131st to 138th amino acids sequence; and a total length of amino acids sequence corresponding to the 131st to 138th amino acids sequence is fourteen or less (preferably 13 or less, more preferably 12 or less). Further, a lower limit of the length of amino acids sequence corresponding to the 131st to 138th amino acids sequence is preferably four or more. The peptide-presenting protein of the present invention may be one wherein one or plural amino acids are deleted, substituted, inserted, and/or added in the first to 130th amino acids sequence or the 139th to 238th amino acids sequence in the amino acid sequence of SEQ ID NO: 54, and exhibiting a fluorescence generation activity.

More preferably, in the peptide-presenting protein of the present invention, a peptide is presented in a presentation region consisting of the 131st to 137th amino acids in the amino acid sequence of the SEQ ID NO: 54, and the peptide consisting of four to thirteen amino acids (preferably 4 to 12 amino acids) is inserted in the 131st to 137th amino acids sequence, or substituted for any of 1 to 7 amino acid(s) sequence in the 131st to 137th amino acids sequence; and a total length of amino acids sequence corresponding to the 131st to 137th amino acids sequence is thirteen or less (preferably 12 or less). Further, a lower limit of the length of an amino acids sequence corresponding to the 131st to 137th amino acids sequence is preferably four or more. The peptide-presenting protein of the present invention may be one wherein one or plural amino acids are deleted, substituted, inserted, and/or added in the first to 130th amino acids sequence or the 139th to 238th amino acids sequence in the amino acid sequence of SEQ ID NO: 54, and exhibiting a fluorescence generation activity.

In connection to this, the presented peptide having a shorter length is preferable, because an aggregation of the protein may be suppressed. Further, as to the presentation region, the region consisting of the 131st to 138th amino acids, or the region consisting of the 131st to 137th amino acids is preferable, because an aggregation of the protein may be suppressed, compared to the region consisting of the 131st to 139th amino acids.

### <<Protein for Presenting a Peptide>>

The protein for presenting a peptide of the present invention can present a peptide in a presentation region consisting of the 131st to 139th amino acids in the amino acid sequence of the SEQ ID NO: 54, and the peptide consisting of four to fifteen amino acids (preferably 4 to 14 amino acids, more preferably 4 to 13 amino acids, most preferably 4 to 12 amino acids) is inserted in the 131st to 139th amino acids sequence, or substituted for any of 1 to 9 amino acid(s) sequence in the 131st to 139th amino acids sequence; and a total length of amino acids sequence corresponding to the 131st to 139th amino acids sequence is fifteen or less (preferably 14 or less, more preferably 13 or less, most preferably 12 or less). Further, a lower limit of the length of an amino acids sequence corresponding to the 131st to 139th amino acids sequence is preferably five or more.

Further, the protein for presenting a peptide of the present invention may be one wherein one or plural amino acids are deleted, substituted, inserted, and/or added in the first to 130th amino acids sequence or the 140th to 238th amino acids sequence in the amino acid sequence of SEQ ID NO: 54, and exhibiting a fluorescence generation activity. The protein consisting of the amino acid sequence of the SEQ ID NO: 54 is the sfGFP described in Non-patent literature 2. However, GFP-based proteins can be used as the scaffold protein for inserting the peptide in the protein for presenting a peptide of the present invention. That is to say, GFP or GFP-based variant protein derived from GFP can be used without limitation. As a concrete scaffold protein, there may be mentioned GFP, YFP, CFP, BFP, or RFP.

Preferably, the protein for presenting a peptide of the present invention can present a peptide in a presentation region consisting of the 131st to 138th amino acids in the amino acid sequence of the SEQ ID NO: 54, and the peptide consisting of four to fourteen amino acids (preferably 4 to 13 amino acids, more preferably 4 to 12 amino acids) is inserted in the 131st to 138th amino acids sequence, or substituted for any of 1 to 8 amino acid(s) sequence in the 131st to 138th amino acids sequence; and a total length of amino acids sequence corresponding to the 131st to 138th amino acids sequence is fourteen or less (preferably 13 or less, more preferably 12 or less). Further, a lower limit of the length of an amino acids sequence corresponding to the 131st to 138th amino acids sequence is preferably five or more. The protein for presenting a peptide of the present invention may be one wherein one or plural amino acids are deleted, substituted, inserted, and/or added in the first to 130th amino acids sequence or the 139th to 238th amino acids sequence in the amino acid sequence of SEQ ID NO: 54, and exhibiting a fluorescence generation activity.

More preferably, the protein for presenting a peptide of the present invention can present a peptide in a presentation region consisting of the 131st to 137th amino acids in the amino acid sequence of the SEQ ID NO: 54, and the peptide consisting of four to thirteen amino acids (preferably 4 to 12 amino acids) is inserted in the 131st to 137th amino acids sequence, or substituted for any of 1 to 7 amino acid(s) sequence in the 131st to 137th amino acids sequence; and a total length of amino acids sequence corresponding to the 131st to 137th amino acids sequence is thirteen or less (preferably 12 or less). Further, a lower limit of the length of an amino acids sequence corresponding to the 131st to 137th amino acids sequence is preferably five or more. The protein for presenting a peptide of the present invention may be one wherein one or plural amino acids are deleted, substituted, inserted, and/or added in the first to 130th amino acids sequence or the 139th to 238th amino acids sequence in the amino acid sequence of SEQ ID NO: 54, and exhibiting a fluorescence generation activity.

The term “peptide-presenting protein” as used herein means, for example, a protein presented a peptide in the region of the 131th to 139th amino acids of the sfGFP. For example, it means an sfGFP in which any peptides for peptide screening are previously inserted therein. As a concrete embodiment, it means a protein expressed from the vector included in the peptide library of the present invention.

On the other hand, the term “protein for presenting a peptide” as used herein means, for example, a protein which is designed so that a peptide can be presented in the region of the 131st to 139th amino acids of the sfGFP. For example, it means an sfGFP in which any peptides for peptide screening are not inserted yet, although any peptides for peptide screening can be inserted therein. As a concrete embodiment, it means a protein translated from the vector which is designed so that a peptide can be inserted in the region of the 131st to 139th amino acids of the sfGFP. Therefore, a polynucleotide encoding the protein for presenting a peptide preferably has restriction enzyme site(s) for inserting nucleotides encoding any peptides.

The region of the 131st to 139th amino acids may be preferably a region the 131st to 138th amino acids, more preferably a region the 131st to 137th amino acids. This is because, when the length of the region is shorter, the peptide-presenting protein has a tendency to easily dissolve. Further, the length of the inserted peptide is preferably 4 to 15, more preferably 4 to 13, more preferably 4 to 13, most preferably 4 to 12. This is because, when the length of the peptide is shorter, the peptide-presenting protein has a tendency to easily dissolve.

(Presentation Region: 131st to 139th Amino Acids Sequence)

A sequence of 131st to 139th amino acids in the amino acid sequence of sfGFP is KEDGNLGH (SEQ ID NO: 55). When the region of the nine amino acids sequence is substituted with a peptide consisting of 4 to 15 (preferably 4 to 14) amino acids sequence, a structural flexibility of the peptide is restricted so as to be presented on the sfGFP in a stable structure. Thus, peptide(s) highly specific to a target molecule can be screened by using the peptide-presenting proteins as the peptide library.

When the presentation region is the 131st to 139th amino acids sequence, the inserted or substituted amino acids sequence is 4 to 15 amino acids, preferably 4 to 14 amino acids, and may include a part of the 13st to 139th amino acids sequence. Thus, 4 to 6 amino acids sequence may be inserted without deletion of the 131st to 139th amino acids sequence. Further, any one of the number of 1 to 9 amino acids sequence in the 131st to 139th amino acids sequence are deleted, and a exogenous amino acid(s) sequence may be inserted so that the total number of the amino acids is 5 to 15, preferably 5 to 14.

Preferably, the presentation region is the 131st to 138th amino acids sequence. In this case, the inserted or substituted amino acids sequence is 4 to 14 amino acids, preferably 4 to 13 amino acids, and may include a part of the 131st to 138th amino acids sequence. Thus, 4 to 6 amino acids sequence may be inserted without deletion of the 131st to 138th amino acids sequence. Further, any one of the number of 1 to 8 amino acids sequence in the 131st to 138th amino acids sequence are deleted, and a exogenous amino acid(s) sequence may be inserted so that the total number of the amino acids is 4 to 14, preferably 4 to 13.

Further, in order to efficiently construct the peptide library, amino acid sequences of both ends of the inserted or substituted peptide is preferably one translated from base sequences containing restriction enzyme sites. The restriction enzyme sites for using for inserting the peptide is not limited. However, for example, BamHI, EcoRI, ScaI, SalI, SphI, PstI, NcoI, NheI, SspI, NotI, SacI, PvuII, MfeI, HindIII, SbfI, EagI, EcoRV, AvrII, BstXI, PciI, HpaI, AgeI, BsmBI, BspQI, SapI, KpnI, or BsaI can be used. For example, the amino acid sequences of both ends of the inserted or substituted 4 to 14 peptides may be amino acid sequence translated from nucleotides containing the base sequence recognized by the above restriction enzyme.

In connection with this, the peptide translated from the restriction enzyme site is sometimes referred to as a linker peptide.

(Deletion, Substitution, Insertion, and/or Addition of Amino Acid(s))

As mentioned above, the GFP-based proteins can be used as the scaffold protein of the peptide-presenting protein and the protein for presenting a peptide, but the scaffold proteins may be proteins wherein one or plural amino acids are deleted, substituted, inserted, and/or added in the amino acid sequence encoding the GFP-based proteins, and the proteins exhibit a fluorescence generation activity.

The term “one or plural amino acids are deleted, substituted, inserted, and/or added in the amino acid sequence” means the GFP-based protein itself or the GFP-based protein modified by the known methods such as site-directed mutagenesis, several naturally occurring amino acids substitution, or the like. The number of modified amino acids is preferably 1 to 15, more preferably 1 to 10, more preferably 1 to 5, more preferably 1 to 3, most preferably 1.

Preferably, examples of the modified amino acids sequence in the polypeptide according to the present invention may be an amino acids sequence having one or plural conservative substitutions.

The term “conservative substitutions” used herein means that one or plural amino acid residues contained in a protein are replaced with different amino acids having similar chemical properties. As for the conservative substitution, there may be mentioned, for example, a substitution of a hydrophobic residue for another hydrophobic residue, or a substitution of a polar residue for another polar residue having the same charge. Amino acids which have similar chemical properties and can be conservatively substituted with each other are known to those skilled in the art. More particularly, as for nonpolar (hydrophobic) amino acids, there may be mentioned, for example, alanine, valine, isoleucine, leucine, proline, tryptophan, phenylalanine, or methionine. As for polar (neutral) amino acids, there may be mentioned, for example, glycine, serine, threonine, tyrosine, glutamine, asparagine, or cysteine. As for basic amino acids having a positive charge, there may be mentioned, for example, arginine, histidine, or lysine. As for acidic amino acids having a negative charge, there may be mentioned, for example, aspartic acid or glutamic acid. In the “conservative substitution”, an original amino acid may be substituted with an amino acid residue which is not chemically similar thereto, but structurally similar thereto. For example, it includes a substitution of alanine (A) for aspartic acid (D), a substitution of alanine (A) for asparagine (E), and a substitution of phenylalanine (F) for tyrosine (Y).

### (Fusion Protein)

In the fusion protein of the present invention, the peptide-presenting protein, or the protein for presenting a peptide of the present invention is fused with a surface protein.

The term “surface protein” used herein means a protein which is presented on a surface of the host, as mentioned below. For example, when the host is a phage, it means a surface protein of the phage. Further, when the host is bacteria, it means a surface protein of the bacteria. Furthermore, when the host is yeast, it means a surface protein of the yeast; and when the host is animal cells, it means a surface protein of the animal cells.

The peptide-presenting protein can be presented on the surface of hosts, by fusing the peptide-presenting protein or protein for presenting a peptide with the surface protein of the hosts. Thus, when the peptide library of the present invention is subjected to an affinity selection (screening) of the peptide of interest, the peptide(s) of interest can be easily concentrated.

The surface protein of phage is not limited, but examples include g3p or g8p of M13 phage, G10 of T7 phage.

The surface protein of bacteria, for example Escherichia coli is not limited, but examples include OmpA.

The surface protein of yeast is not limited, but examples include Flo1, α agglutinin, or AGA2.

The surface protein of animal cells is not limited, but examples include human Toll-like receptor 4, EGFR, LDL receptor, angiotensin II receptor, or PDGFR.

### (Polynucleotide)

The polynucleotide of the present invention is one encoding the peptide-presenting protein of the present invention (hereinafter sometimes referred to as peptide-presenting polynucleotide), one encoding the protein for presenting a peptide of the present invention (hereinafter sometimes referred to as polynucleotide for presenting a peptide), or one encoding the fusion protein of the present invention (hereinafter sometimes referred to as fusion polynucleotide). The fusion polynucleotide may contain a nucleotide encoding a linker polypeptide which may exist between the peptide-presenting protein or protein for presenting a peptide, and the surface protein. In connection to this, the base sequence of the SEQ ID NO: 53 is a base sequence of nucleotide encoding the sfGFP.

### (Vector)

The polynucleotide of the present invention is not limited, but can be easily proliferated by being inserted into a vector. Further, it can be easily expressed as the peptide-presenting protein or the fusion protein.

The vector for inserting the polynucleotide is not limited, so long as it can be replicated in host cells. Any vectors such as plasmids, phages, or viruses can be used. For example, there may be mentioned a plasmid for Escherichia coli, such as pBR322, pBR325, pUC118, pUC119, pKC30, or pCFM536, a plasmid for Bacillus subtilis, such as pUB110, a plasmid for yeast, such as pG-1, YEp13, or YCp50, phage DNA such as λgt110, or λZAPII. Further, as the vector for mammalian cells, there may be mentioned a virus DNA such as baculovirus, vaccinia virus, or adenovirus; SV40; or derivatives thereof. The vector contains an initiation site of replication, a selective marker, and a promotor, and if necessary may contain an enhancer, a terminator, a ribosomal binding site, and polyadenylation signal.

For example, when the vector is used as the library of the present invention, M13 phage vector, or T7 phage vector is preferably used. This is because either can be used as a vector for phage display.

### (Host)

The “host” used herein is not limited as long as the vector can be inserted therein and the peptide-presenting protein or the fusion protein can be expressed thereby, and the host includes phages, bacteria, yeasts, insect cells, or animal cells. For example, as the host for the phage vector, there may be mentioned M13 phage, fd phage, or T7 phage.

Further, as the host for the bacteria vector, there may be mentioned Escherichia coli, Streptomyces, or Bacillus subtilis. Furthermore, as the host for the yeast vector, there may be mentioned baker's yeast, or methanol-assimilating yeast. As the host for insect cells, there may be mentioned Drosophila S2, or Spodoptera Sf9. Further, as the host for animal cells, there may be mentioned CHO, COS, BHK, 3T3, or C127.

### [2] LIBRARY

The library of the present invention is a library for peptide presentation, and the peptides presented thereon have random amino acid sequences. The library for peptide presentation can be used for screening peptide(s) capable of binding to a target molecule. Therefore, the presented peptides preferably contain a number of (many kinds of) peptides consisting of random amino acid sequences. Further, the library of the present invention contains the above host, and thus includes a phage display library, or a cell display library of bacteria, yeast, insect cells, or animal cells.

A length of the presented peptide provided for screening using the library of the present invention is not particularly limited, but for example, 4 to 15, preferably 4 to 14, more preferably 4 to 13.

### [3] METHOD FOR CONSTRUCTING LIBRARY

The method for constructing a library comprises the steps of: (1) inserting a nucleotide encoding a peptide having 4 to 15 amino acids (preferably 4 to 14) into the vector containing polypeptide encoding the protein for presenting a peptide of the present invention to obtain an expression vector, wherein the peptide is inserted in the 131st to 139th amino acids sequence of the presentation region, or substituted for any one of 1 to 9 amino acid(s) sequence in the 131st to 139th amino acids sequence of the presentation region; and a total length of amino acids sequence corresponding to the 131st to 139th amino acids sequence is 15 or less (preferably 14 or less), and (2) introducing the vector into a host. In particular, the library is the library for peptide presentation, and can present any peptides.

Preferably, the method for constructing a library comprises the steps of: (1) inserting a nucleotide encoding a peptide having 4 to 14 amino acids (preferably 4 to 13) into the vector containing polypeptide encoding the protein for presenting a peptide of the present invention to obtain an expression vector, wherein the peptide is inserted in the 131st to 138th amino acids sequence of the presentation region, or substituted for any one of 1 to 8 amino acid(s) sequence in the 131st to 138th amino acids sequence of the presentation region; and a total length of amino acids sequence corresponding to the 131st to 138th amino acids sequence is 14 or less (preferably 13 or less), and (2) introducing the vector into a host.

The vector used for the method for constructing a library contains polypeptide encoding the protein for presenting a peptide, and polynucleotides encoding any polypeptides can be easily inserted therein by using a restriction enzyme site.

A length of the polypeptide encoded by the inserted nucleotides is not particularly limited, but for example, 4 to 15, preferably 4 to 14, more preferably 4 to 13.

In the vector construction and the vector introduction into the host, the known methods can be used. For example, when the host is the phage, library genes of peptides to be presented are inserted into vector arms by a ligation. The resulting ligation solution is mixed with a packaging solution containing phage proteins to form phage particles. A phage library can be constructed by infected Escherichia coli such as BLT5403 with the phage particles.

### (Library Gene)

Genes for inserting into a library can be prepared by known methods.

For example, a codon encoding any amino acids is substituted with NNK codons (wherein N is any one of A, G, C, or T, and K is any one of G or T) encoding random amino acids in a site-specific manner. The substitution is carried out by chemically synthesizing oligo DNAs containing the above region and inserting the same using a restriction enzyme; or using a method for replicating the full length vector using PCR.

### [4] SCREENING METHOD

The screening method of the present invention comprises the steps of: (1) bringing into contact the above peptide library with the target molecule, and (2) recovering hosts binding to the target molecule. Preferably, the screening method further comprises (3) the step of proliferating the recovered hosts. In the screening method of the present invention, the contact step (1), preferably the host-recovering step (2), and the proliferating step (3) are repeated, to thereby concentrate the peptides binding to a target molecule. The contact between the target molecule and the peptide library in the contact step (1) can be carried out by immobilizing the target molecule to an insoluble carrier such as wells or dishes.

The screening method of the present invention may be carried out according to known methods, except that the library of the present invention is used. For example, the screening for the phage library using phage as the host may be performed as follows. The phage library is brought into contact with the target molecule immobilized to a plate or the like so as to react. Phages which are not bound to the target molecule are removed by washing. Phages which are bound to the target molecule are recovered. Then, Escherichia Coli is infected with the phages and is lysed to thereby proliferate the phages. A supernatant after bacteriolysis or purified phages are further reacted with the immobilized target molecule to thereby concentrate the phages wherein a binding molecule specific to the target molecule is presented (Panning). Finally, the phages are cloned and the peptide presented thereon is determined by a sequence method.

The obtained peptide can bind with the target molecule. In the screening method of the present invention, as the peptides are presented on the protein for presenting a peptide, the structure of the peptides is stable. Therefore, the peptides highly specific to the target molecule can be screened.

### EXAMPLES

The present invention now will be further illustrated by, but is by no means limited to, the following Examples.

### Reference Example 1

### Construction of sfGFP Expression Vector

In this reference example, a vector expressing sfGFP was constructed.

A PCR was carried out by a KOD FX (TOYOBO), using a pGEX-6P-3-PTD-3-ODD-EGFP vector (Kizaka-Kondoh et al., Clin. Cancer. Res., 2009, vol. 15, p. 3433-3441) having a gene of Enhanced GFP (EGFP) as a template, and primers (SEQ ID NO: 1 and 2) (Table 2), to obtain a EGFP gene fragment of 0.7 kb length in which restriction enzyme (Bam HI or Eco RI) sites were added to both ends. The fragment was digested with restriction enzymes Bam HI and EcoRI and it was ligated with a cloning pGEM-3Zf(+) vector (Promega) which was preliminarily digested with Bam HI and EcoRI. A pGEM-EGFP vector wherein the EGFP gene was inserted, was obtained.

A PCR was carried out, using the pGEM-EGFP vector as a template, primers (SEQ ID NO: 3 to 16) (Table 2), and PfuUltra High fidelity DNA Polymerase (Agilent Technologies) to obtain a pGEM-sfGFP vector wherein the sfGFP gene which has site-directed mutations for substituting 9 amino acids was inserted. The vector was digested with Bam HI and Eco RI to obtain a Bam HI-Eco RI fragment. The fragment was bound to a BamHI and Eco RI site of a pGEX-6P-3 vector for expression of GST fusion protein (GE Healthcare) to obtain a pGEX-sfGFP vector. The pGEX-sfGFP has a length of 5.6 kbp, and has in order of a base sequence of GST structural gene and a base sequence of sfGFP at the downstream of tac promoter. Therefore, the pGEX-sfGFP vector intracellularly expresses a fusion protein containing in order of GST and sfGFP in the Escherichia Coli.

In these examples, peptide-presenting proteins wherein a caspase-3 recognizing peptide was inserted in the 131st to 139th amino acids sequence, were prepared.

A PCR was carried out using the pGEX-sfGFP vector constructed in the reference example 1 as a template, primers (SEQ ID NO: 7, 18, 23-28, 31-46, 58-59, and 64-71) (Table 2), and PfuUltra High fidelity DNA Polymerase to obtain a pGEX-m1 vector (Example 1), pGEX-m4 to pGEX-m6 vectors (Examples 2 to 4), pGEX-m10 to pGEX-m22 vectors (Examples 5 to 17) for expressing sfGFP in which the caspase-3 recognizing peptide (DEVD) is inserted in a site-specific manner. Lengths and positions of substituted peptides are shown in Table 1. The peptide-presenting proteins obtained in Example 16 (m10) and Example 17 (m22) were easily insolubilized.

### Comparative Examples 1 to 5

In these comparative examples, peptide-presenting proteins wherein the caspase-3 recognizing peptide was inserted in an amino acid sequence other than the 131st to 139th amino acids sequence, were prepared.

The procedure described in Example 1 was repeated except that primers having base sequences of SEQ ID NO: 19 and 20 (Comparative Example 1), SEQ ID NO: 21 and 22 (Comparative Example 2), SEQ ID NO: 29 and 30 (Comparative Example 4), SEQ ID NO: 60 and 61 (Comparative Example 4), and SEQ ID NO: 62 and 63 (Comparative Example 5) were used, to obtain a pGEX-m2 vector (Comparative Example 1), a pGEX-m3 (Comparative Example 2), a pGEX-m7 (Comparative Example 3), a pGEX-m8 (Comparative Example 4), and a pGEX-m9 (Comparative Example 5). Lengths and positions of substituted peptides are shown in Table 1.

TABLE 1

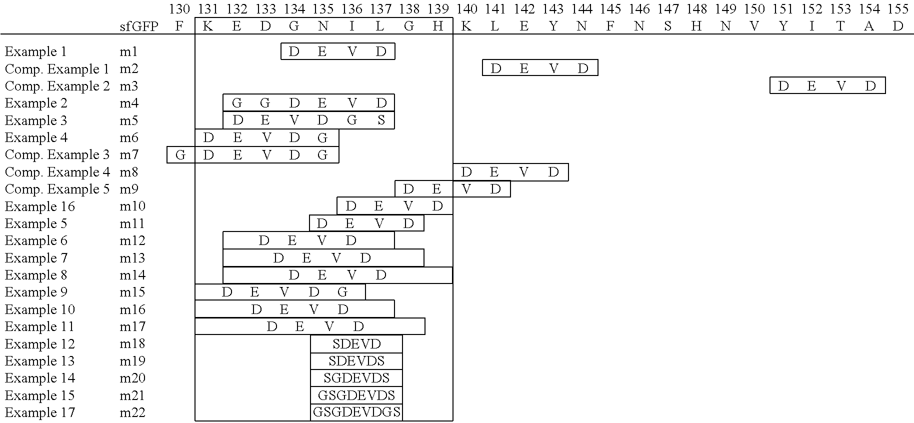

### <<Expression and Purification of Peptide-Presenting Protein>>

Peptide-presenting proteins were expressed and purified using the vectors obtained in Examples 1 to 17, and Comparative Examples 1 to 5.

An Escherichia Coli BL21-CodonPlus (DE3) strain (Agilent Technologies) was transformed with the respective pGEX-sfGFP vectors, i.e. pGEX-m1 to pGEX-m22 vectors by a heat shock method. Each of the Escherichia Coli containing the expression vectors was cultured in a 50 mL of LBA medium (containing 0.01% ampicillin, 1% Bacto Tryptone, 0.5% Bacto Yeast extract, 1% NaCl) at 37° C., so as to be 0.5 of O.D.600. Then, Isopropyl β-D-1-thiogalactopyranoside was added to the medium to give 0.1 mM of the final concentration, and the Escherichia Coli was cultured at 20° C. over night, to obtain an Escherichia Coli wherein a GST fusion protein was expressed.

Each of the Escherichia Coli in which the protein was expressed was collected by centrifugation and was re-suspended in 20 mL of phosphate buffer solution (PBS). The obtained bacteria were crushed by three freeze-thaw, and ultrasonic fragmentation, Subsequently, Triton x-100 was added thereto to give 1% of the final concentration, and the whole was allowed to stand on ice for 30 minutes. Next, the solution containing bacteria were centrifuged at 5000 rpm for 10 minutes at 4° C., and the supernatant containing the protein was collected. Then, the collected solution was added to GST agarose beads washed by PBS, and the GST fusion protein was adsorbed thereto while gently rotating at 4° C. over night. The agarose beads were washed by PBS, and then washed by Prescission Cleavage Buffer (0.05M Tris-HCl (pH7.0), 0.15M NaCl, 1 mM EDTA, 1 mM DTT). Subsequently, in order to cleave the fusion protein to GST and sfGFP or sfGFP variants (sfGFP-m1 to sfGFP-m22), 1 mL of Prescission Cleavage Buffer and 5 μL of Prescission Protease (GE Healthcare) were added thereto. The whole was reacted at room temperature for 4 hours, and then reacted at 4° C. over night. Next, the reactant was centrifuged at 3000 rpm for 15 minutes at 4° C., to collect a supernatant. A buffer of the supernatant was substituted by an ultrafiltration membrane capable of cutting 10 kDa, to obtain about 5 mg of peptide-presenting protein (sfGFP-m1 to m9 and sfGFP-m11 to m21) (hereinafter, the peptide-presenting proteins obtained from pGEX-m1 to m9 and pGEX-m11 to m21 are sometimes referred to as m1 to m9 and m11 to m21, respectively). The sfGFP-m10 and sfGFP-m22 were expressed in inclusion bodies.

### <<Cleavage of Peptide-Presenting Protein by Caspase-3>>

In the peptide-presenting proteins obtained from pGEX-m1 to m9 and m11 to m21, the caspase-3 recognizing peptide (DEVD) was inserted. In order to study the structural flexibility of the peptide-presenting protein, the peptide-presenting proteins were cleaved by caspase-3.

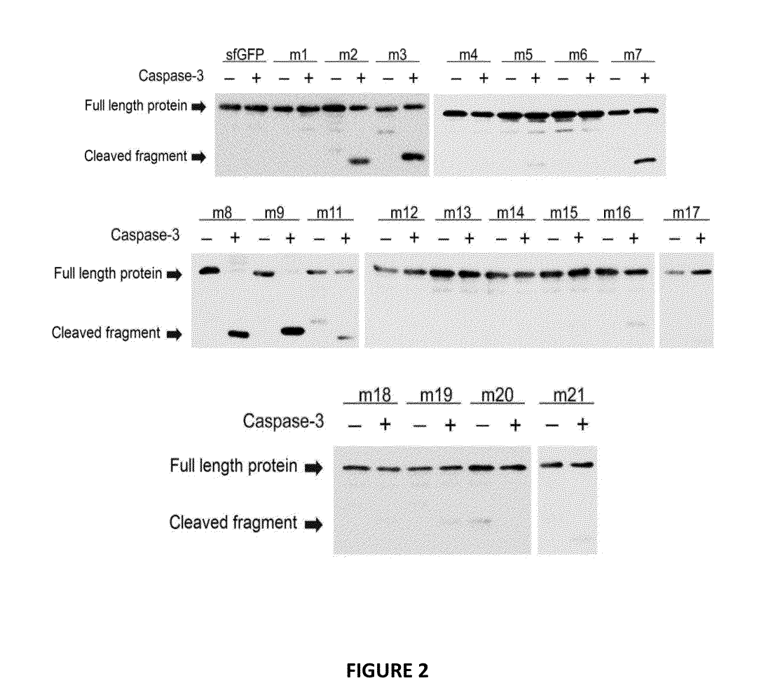

10 μM of the peptide-presenting proteins m1 to m9 and m11 to m21 were reacted with 4 mU/mL of human caspase-3 (Sigma-Aldrich) at 37° C. for 24 hours in a caspase reaction buffer (20 mM pipes (pH7.2), 100 mM NaCl, 0.1% CHAPS, 10% sucrose, 10 mM DTT). The sfGFP was used as a control. The reacted solutions were subjected to a 15% acrylamide gel electrophoresis, and transferred to a Hybond ECL membrane (GE Healthcare) at 20V constant voltage for 1 hour. Next, after blocking with a 5% skim milk, the membrane was reacted with a first antibody (rabbit polyclonal anti-GFP antibody) at 4° C. over night. Then, the membrane was reacted with a second antibody (HRP conjugated anti-rabbit IgG antibody) at room temperature for 1 hour, and cleaved fragments were detected by adding ECL prime (GE Healthcare). As a result, m2 (Comparative Example 1), m3 (Comparative Example 2), m7 (Comparative Example 3), m8 (Comparative Example 4), and m9 (Comparative Example 5) were cleaved by caspase-3. On the other hand, m1 (Example 1), m4 (Example 2), m5 (Example 3), m6 (Example 4), m11 (Example 5), m12 (Example 6), m13 (Example 7), m14 (Example 8), m15 (Example 9), m16 (Example 10), m17 (Example 11), m18 (Example 12), m19 (Example 13), m20 (Example 14), and m21 (Example 15) were hardly cleaved (Table 1 and FIG. 2). These results show that when the caspase-3 recognizing peptides having amino acid lengths of 5 to 14 were inserted in the region of the 131st to 139th amino acids of the sfGFP, they could not bind to caspase-3, and when the caspase-3 recognizing peptides were inserted in regions other than the above region, they could bind to caspase-3 That is to say, the peptides inserted in the region of the 131st to 139th amino acids were structurally fixed, and it is considered that the structural flexibility thereof was limited.

### <<Fluorescence Intensity of Peptide-Presenting Protein>>

An influence on a fluorescence intensity of peptide-presenting protein due to the insertion of the peptides was investigated.

The peptide-presenting proteins (m1 to m9 and m11 to m21) were dissolved in a buffer (20 mM Pipes (pH7.2), 100 mM NaCl, 0.1% CHAPS, 10 sucrose, 10 mM DTT) to give 10 μM of the concentration, and the fluorescence (excitation wavelength/fluorescence wavelength=480/510 nm) was measured. The fluorescence was observed in the peptide-presenting proteins of m1 (Example 1), m4 (Example 2), m5 (Example 3), m6 (Example 4), m11 (Example 5), m12 (Example 6), m13 (Example 7), m14 (Example 8), m15 (Example 9), m16 (Example 10), m17 (Example 11), m18 (Example 12), m19 (Example 13), m20 (Example 14), and m21 (Example 15) wherein the structural flexibility of the peptides was limited, but the fluorescence was quenched in the peptide-presenting proteins of m2 (Comparative Example 1), m3 (Comparative Example 2), m7 (Comparative Example 3), m8 (Comparative Example 4), m9 (Comparative Example 5) (Table 1 and Table 3). That is, the results showed that the fluorescence of GFP was maintained, when the caspase-3 recognizing peptides having amino acids length of 5 to 14 were inserted in the region of the 131st to 139th amino acids of the sfGFP.

### Analytical Example 1

### Analysis of Conformational Fluctuation of K131-H139 Region

If the structural flexibility of the peptides inserted into the prepared peptide-presenting protein was limited, it was considered that a conformational fluctuation of the K131-H139 region in a molecular dynamics simulation was reduced.

The molecular dynamics simulations of sfGFP, m1 (Example 1), m6 (Example 4), and m19 (Example 13), which were not easily cleaved by caspase-3, were carried out. As a control, the m7 (Comparative Example 3) which was cleaved by caspase-3, was used. Initial model of these proteins were prepared from a crystallographic structure of sfGFP (PDB ID:2B3P). For simplicity of calculation, two amino acids (65T and 66Y) which constitute fluorophore were substituted to glycine residues. Thermodynamically stable structures thereof were calculated from the initial model using a force field of Amber 11 program package (Amber). In the molecular dynamics simulation, a structure for 10 nanoseconds in a GB/SA solvent model was calculated using a Sander module of Amber 11, and then the fluctuation was evaluated by using a structure of 4.5 to 10 nanoseconds which reaches a state of equilibrium.

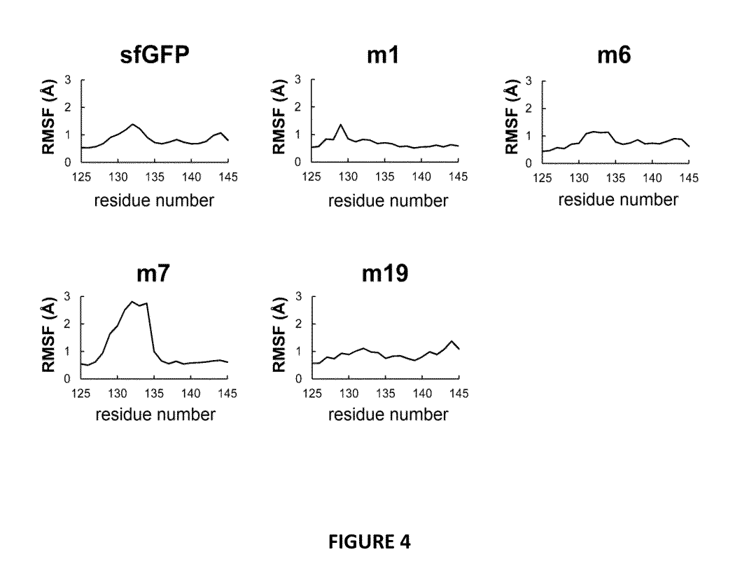

The results are shown in FIG. 4. A longitudinal axis is a magnitude of fluctuation of each residue at 4.5 to 10 nanoseconds in the simulation, and an abscissa axis is the residue number. The fluctuations in the K131-H139 region of the sfGFP, m1 (Example 1), m6 (Example 4), m19 (Example 13) were within 0.1 nanometers at the most residues. However, the fluctuations in the K131-H139 region of the m7 (Comparative Example 3) was increased up to about 0.3 nanometers, and the results showed that the K131-H139 region may fluctuate widely. Thus, it was revealed that the variants which were not easily cleaved by caspase-3 have little fluctuation, and the structural flexibility of the peptide inserted therein was limited.

### Examples 16 to 20

In this example, five peptides which bind to a breast cancer marker protein, i.e. HER2 according to reports were inserted in the 131st to 139th amino acids sequence region of sfGFP, to obtain expression vectors and purified proteins.

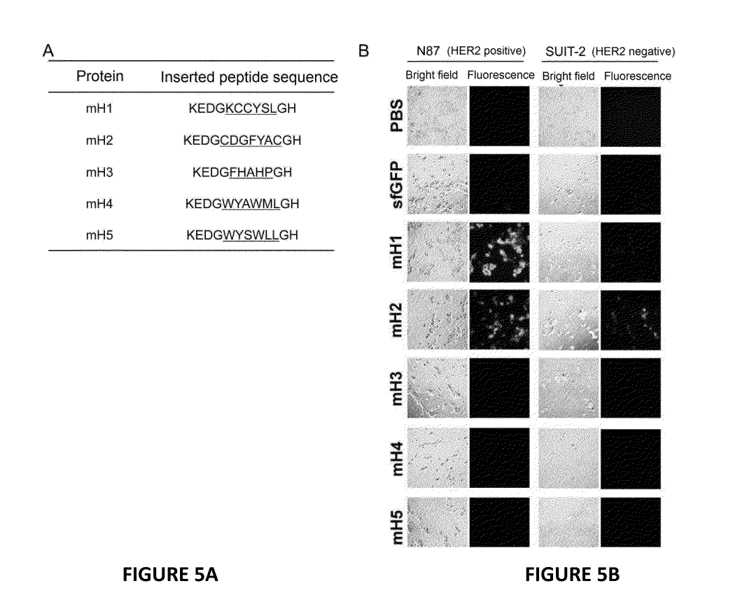

The procedures described in Example 1 were repeated except that primers of SEQ ID NO: 47 and 48 (Example 16), SEQ ID NO: 49 and 50 (Example 17), SEQ ID NO: 51 and 52 (Example 18), SEQ ID NO: 72 and 73 (Example 19), or SEQ ID NO: 74 and 75 (Example 20) used as primers, to obtain pGEX-mH1 vector (Example 16), pGEX-mH2vector (Example 17), pGEX-mH3vector (Example 18), pGEX-mH4vector (Example 19), or pGEX-mH5vector (Example 20). Lengths and positions of substituted peptides are shown in FIG. 5(A). A sequence of mH1 is “KEDGKCCYSLGH” (SEQ ID NO:55), a sequence of mH2 is “KEDGCDGFYACGH” (SEQ ID NO:56), a sequence of mH3 is “KEDGFHAHPGH” (SEQ ID NO:57), a sequence of mH4 is “KEDGWYAWMLGFF” (SEQ ID NO:76), and a sequence of mH is “KEDGWYSWLLGH” (SEQ ID NO:77).

Further, the procedures described in the item of “Expression and purification of peptide-presenting protein” were repeated except that the pGEX-mHlvector, pGEX-mH2 vector, pGEX-mH3 vector, pGEX-mH4 vector, and pGEX-mH5 vector were used to obtain peptide-presenting proteins (hereinafter, the peptide-presenting proteins obtained from the pGEX-mH1 to mH5 vectors are sometimes referred to as mH1 to mH5, respectively).

The HER2 protein which may frequently be overexpressed in breast cancer cells, is useful for a target for treatment or imaging of breast cancer, and a peptide which can function in a living body of a mouse was reported. The peptide, mH1, was screened from a phage library, and has an S-S bond between neighboring cysteines, and the construction thereof was partially limited (Karasseva et al., J. Protein Chem., 2002, vol. 21, p. 287-296). The peptide, mH2, was designed on the basis of a binding surface of an antibody binding to HER2, and exhibits the binding activity under the condition that the peptide is circularized and the structure thereof is limited (Park et al., Nat. Biotechnol., 2000, vol. 18, p. 194-198). The peptide, mH3, was selected by in silico screening from an unstructured peptide library (Nakajima et al., Breast Cancer, 2008, vol. 15, p. 65-72). The peptides, mH4 and mH5, were screened concomitantly with the peptide, mH1, and the structures thereof were not limited. It was preliminarily reported that the mH1 and mH2 can function in a living body of a mouse (Deutscher S L, Chem. Rev., 2010, vol. 110, p. 3196-3211).

### <<Binding to Cancer Cells, of Peptide-Presenting Protein Wherein the HER2 Peptide is Inserted>>

2.5×10⁴ of HER2 positive N87 stomach cancer cell or HER2 negative SUIT-2 pancreatic cancer cell was cultured in Swell slide chamber culture plate for 16 hours. The cells were fixed by being reacted with a 4% paraformaldehyde phosphate buffer for 15 minutes, washed by PBS solution, and blocked using 3% bovine serum albumin/TBST for 15 minutes. Then, after adding 2 μM of peptide-presenting protein (mH1, mH2, mH3, mH4, or mH5), the cells were incubated at 4° C. for 16 hours, and binding activities were evaluated by a fluorescence microscope observation. As a result, it was revealed that mH1 and mH2 specifically bind to N87 cell, but mH3, mH4, and mH5 may not bind thereto (FIG. 4(B)). That is, when the peptides which were screened under the conditions that the structures thereof were not limited, were inserted into the vector, the peptides exhibit a high binding property as well. However, when the peptides which were screened under the conditions that the structure of peptides can freely vary, the peptides cannot bind thereto. It was already known that the former can function in a living body of a mouse. Therefore, the results showed that the peptides which have high specificity to target and function in a living body, can be easily and high-sensitively screened by using the protein wherein the peptides were inserted in the K131-H139 region of sfGFP, and evaluating the same.

TABLE 2-1
SEQ ID No: 1:
5′-TCTAGAGGATCCATGGTGAGCAAGGGCGAGGAG-3′
SEQ ID No: 2:
5′AGGGCGAATTCTTAATGGTGATGGTGATGATGGCTGCCCTTGTACAG
CTCGTCCATGCC-3′
SEQ ID No: 3:
5′-CACAAGTTCAGCGTGCGCGGCGAGGGCGAGGGCGATGCCACCAACG
GCAAGCTGACCCTG-3′
SEQ ID No: 4:
5′-CAGGGTCAGCTTGCCGTTGGTGGCATCGCCCTCGCCCTCGCCGCGC
ACGCTGAACTTGTG-3′
SEQ ID No: 5:
5′-CAGGAGCGCACCATCAGCTTCAAGGACGACGGCACCTACAAGACCC
GCGCC-3′
SEQ ID No: 6:
5′-GGCGCGGGTCTTGTAGGTGCCGTCGTCCTTGAAGCTGATGGTGCGC
TCCTG-3′
SEQ ID No: 7:
5′-CTGAGCACCCAGTCCGTGCTGAGCAAAGACCCC-3′
SEQ ID No: 8:
5′-GGGGTCTTTGCTCAGCACGGACTGGGTGCTCAG-3′
SEQ ID No: 9:
5′-CAAGCTGGAGTACAACTTCAACAGCCACAACGTC-3′
SEQ ID No: 10:
5′-GACGTTGTGGCTGTTGAAGTTGTACTCCAGCTTG-3′
SEQ ID No: 11:
5′-CACAACGTCTATATCACCGCCGACAAGCAGAAG-3′
SEQ ID No: 12:
5′-CTTCTGCTTGTCGGCGGTGATATAGACGTTGTG-3′
SEQ ID No: 13:
5′-GAAGAACGGCATCAAGGCGAACTTCAAGATCCGC-3′
SEQ ID No: 14:
5′-GCGGATCTTGAAGTTCGCCTTGATGCCGTTCTTC-3′
SEQ ID No: 15:
5′-CAAGATCCGCCACAACGTGGAGGACGGCAGCGTG-3′
SEQ ID No: 16:
5′-CACGCTGCCGTCCTCCACGTTGTGGCGGATCTTG-3′
SEQ ID No: 17 (m1, Example 1):
5′-GATGAAGTTGATGAGTACAACTTCAACAGC-3′
SEQ ID No: 18 (m1, Example 1):
5′-CAGGATGTTGCCGTCCTC-3′
SEQ ID No: 19 (m2, Comp. Example 1):
5′-AACATCCTGGGGCACAAGGATGAGGTTGATTTCAACAGCCACAACG
TC-3′
SEQ ID No: 20 (m2. Comp. Example 1):
5′-ACGTTGTGGCTGTTGAAATCAACCTCATCCTTGTGCCCCAGGATGT
T-3′
SEQ ID No: 21 (m3, Comp. Example 2):
5′-GATGAAGTTGATGACAAGCAGAAGAACGGC-3′
SEQ ID No: 22 (m3, Comp. Example 2):
5′-GACGTTGTGGCTGTTGAA-3′
SEQ ID No: 23 (m4, Example 2):
5′-GGCGGCGATGAAGTTGATGGGCACAAGCTGGAGTAC-3′
SEQ ID No: 24 (m4, Example 2):
5′-CTTGAAGTCGATGCCCTT-3′
SEQ ID No: 25 (m5, Example 3):
5′-GATGAAGTTGATGGCAGCGGGCACAAGCTGGAGTAC-3′
SEQ ID No: 26 (m5, Example 3):
5′-CTTGAAGTCGATGCCCTT-3′
SEQ ID No: 27 (m6, Example 4):
5′-GATGAAGTTGATGGCATC-3′
SEQ ID No: 28 (m6, Example 4):
5′-GAAGTCGATGCCCTTCAG-3′
SEQ ID No: 29 (m7, Comp. Example 3):
5′-GGCGATGAAGTTGATGGCATCCTGGGGCACAAGCTG-3′
SEQ ID No: 30 (m7, Comp. Example 3):
5′-GTCGATGCCCTTCAGCTC-3′
SEQ ID No: 31 (m12, Example 6):
5′-GATGAAGTTGATGGGCACAAGCTGGAGTAC-3′
SEQ ID No: 32 (m12, Example 6):
5′-CTTGAAGTCGATGCCCTT-3′
SEQ ID No: 33 (m13, Example 7):
5′-CACAAGCTGGAGTACAAC-3′
SEQ ID No: 34 (m13, Example 7):
5′-ATCAACTTCATCCTTGAAGTCGATGC-3′
SEQ ID No: 35 (m14, Example 8):
5′-AAGCTGGAGTACAACTTC-3′
SEQ ID No: 36 (m14, Example 8):
5′-ATCAACTTCATCCTTGAAGTCGATGCC-3′
SEQ ID No: 37 (m15, Example 9):
5′-GGCGATGAAGTTGATGGCCTGGGGCACAAGCTG-3′
SEQ ID No: 38 (m15, Example 9):
5′-GAAGTCGATGCCCTTCAG-3′

TABLE 2-2
SEQ ID No: 39 (m16, Example 10):
5′-GATGAAGTTGATGGGCACAAGCTGGAGTAC-3′
SEQ ID No: 40 (m16, Example 10):
5′-GAAGTCGATGCCCTTCAG-3′
SEQ ID No: 41 (m18, Example 12):
5′-TCCGACGAGGTGGACGGGCACAAGCTGGAGTAC-3′
SEQ ID No: 42 (m18, Example 12):
5′-GCCGTCCTCCTTGAAGTCGAT-3′
SEQ ID No: 43 (m19, Example 13):
5′-TCCGACGAGGTGGACTCCGGGCACAAGGTGGAGTAC-3′
SEQ ID No: 44 (m19, Example 13):
5′-GCCGTCCTCCTTGAAGTCGAT-3′
SEQ ID No: 45 (m20, Example 14):
5′-TCCGGGGACGAGGTGGACTCCGGGCACAAGCTGGAGTAC-3′
SEQ ID No: 46 (m20, Example 14):
5′-GCCGTCCTCCTTGAAGTCGAT-3′
SEQ ID No: 47 (mH1, Example 16):
5′-CCGGCGAGTCCGGCGGATCGAGAAGTGGGGCACAAGCTGGAG-3′
SEQ ID No: 48 (mH1, Example 16):
5′-GCCGTCCTCCTTGAAG-3′
SEQ ID No: 49 (mH2, Example 17):
5′-CTTCAAGGAGGACGGCTGCGATGGTTTTTATGCGTGCGGGCACAAG
CTGGAG-3′
SEQ ID No: 50 (mH2, Example 17):
5′-CTCCAGCTTGTGCCCGCACGCATAAAAACCATCGCAGCCGTCCTCC
TTGAAG-3′
SEQ ID No: 51 (mH3, Example 18):
5′-AAATGCTGCTATAGTCTGGGGCACAAGCTGGAG-3′
SEQ ID No: 52 (mH3, Example 18):
5′-GCCGTCCTCCTTGAAG-3′
SEQ ID No: 58 (m22, Example 17):
5′-GGGTCCGGGGACGAGGTGGACGGGTCCGGGCACAAGCTGGAGTA
C-3′
SEQ ID No: 59 (m22, Example 17):
5′-GCCGTCCTCCTTGAAGTCGAT-3′
SEQ ID No: 60 (m8, Comp. Example 4):
5′-AACATCCTGGGGCACGATGAGGTTGATAACTTCAACAGCCACAA
CGTC-3′
SEQ ID No: 61 (m8, Comp. Example 4):
5′-GACGTTGTGGCTGTTGAAGTTATCAACCTCATCGTGGCCCAGGA
TGTT-3′
SEQ ID No: 62 (m9, Comp. Example 5):
5′-GAGTACAACTTCAACAGC-3′
SEQ ID No: 63 (m9, Comp. Example 5):
5′-ATCAACTTCATCCAGGATGTTGCCGTCCTC-3′
SEQ ID No: 64 (m10, Example 16):
5′-GTTGATAAGCTGGAGTACAAC-3′
SEQ ID No: 65 (m10, Example 16):
5′-TTCATCGTTGCCGTCCTCCTT-3′
SEQ ID No: 66 (m11, Example 5):
5′-GTTGATCACAAGCTGGAGTAC-3′
SEQ ID No: 67 (m11, Example 5):
5′-TTCATCGCCGTCCTCCTTGAA-3′
SEQ ID No: 68 (m17, Example 11):
5′-AAGGGCATCGACTTCGATGAAGTTGAT-3′
SEQ ID No: 69 (m17, Example 11):
5′-ATCAACTTCATCGAAGTCGATGCCCTT-3′
SEQ ID No: 70 (m21, Example 15):
5′-GGGGACGAGGTGGACTCCGGGCACAAGCTG-3′
SEQ ID No: 71 (m21, Example 15):
5′-CAGCTTGTGCCCGGAGTCCACCTCGTCCCC-3′
SEQ ID No: 72 (mH4, Example 19):
5′-TGGTACAGCTGGCTGCTGGGGCACAACCTGGAGTAC-3′
SEQ ID No: 73 (mH4, Example 19):
5′-GCCGTCCTCCTTGAAGTC-3′
SEQ ID No: 74 (mH5, Example 20):
5′-TGGTACGCCTGGATGCTGGGGCACAAGGTGGAGTAC-3′
SEQ ID No: 75 (mH5, Example 20):
5′-GCCGTCCTCCTTGAAGTC-3′
SEQ ID No: 76:
KEDGWYAWMLGH
SEQ ID No: 77:
KEDGWYSWLLGH

### Analytical Example 2

### Analysis of Conformational Fluctuation of K131-L137 Region

The molecular dynamics simulations of m12 (Example 6), m15 (Example 9), m16 (Example 10), m19 (Example 13), m21 (Example 15), and m22 (Example 17), which were peptide-presenting proteins having a K131-L137 region of the present invention, were carried out. The sfGFP was used as a positive control. The procedures described in analytical example 1 were repeated except that the m12 (Example 6), m15 (Example 9), m16 (Example 10), m19 (Example 13), m21 (Example 15), and m22 (Example 17) were used.

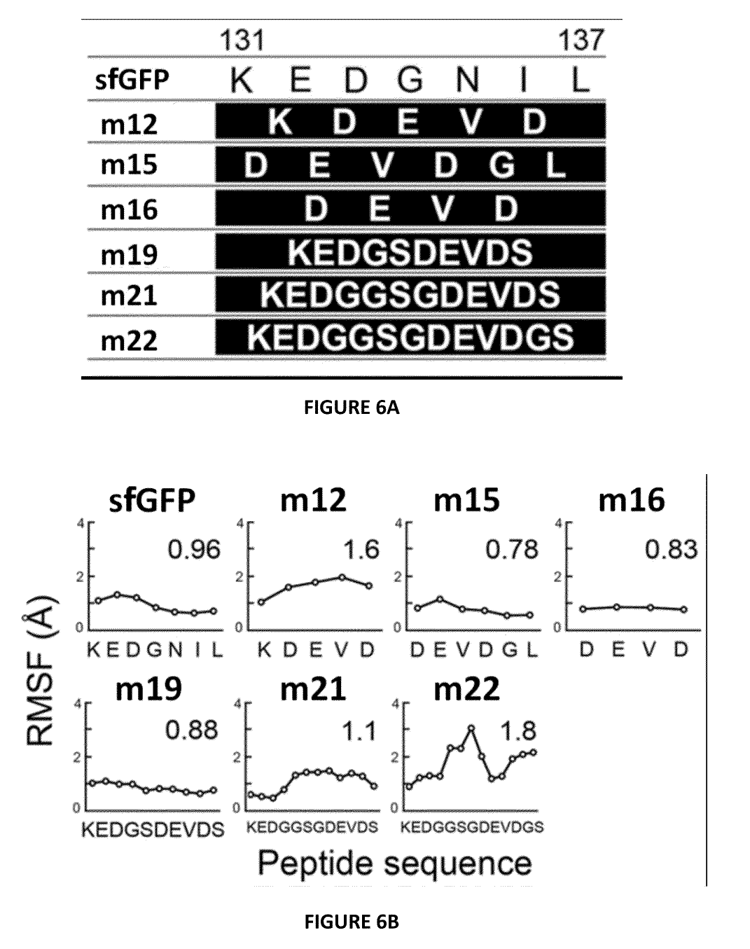

The amino acid sequences are shown in FIG. 6(A) and the analysis results are shown in FIG. 6(B). A longitudinal axis is a magnitude of fluctuation of each residue at 4.5 to 10 nanoseconds in the simulation, and an abscissa axis is the residue number. It was revealed that the variants which were not easily cleaved by caspase-3 have little fluctuation, and the structural flexibility of the peptide inserted therein was limited.

### INDUSTRIAL APPLICABILITY

The peptide-presenting protein and the peptide library using the same can be used as a screening of peptides capable of specifically binding to a target molecule. The peptides screened by using the peptide library of the present invention can be effectively used as an active ingredient for the medicament.

Although the present invention has been described with reference to specific embodiments, various changes and modifications obvious to those skilled in the art are possible without departing from the scope of the appended claims.

1. A peptide-presenting protein wherein a peptide is presented in a presentation region consisting of the 131st to 139th amino acids in the amino acid sequence of the SEQ ID NO: 54, wherein the peptide consisting of four to fifteen amino acids is inserted in the 131st to 139th amino acids sequence, or substituted for any of 1 to 9 amino acid(s) sequence in the 131st to 139th amino acids sequence; and a total length of an amino acid sequence corresponding to the 131st to 139th amino acids sequence is fifteen or less.

2. The peptide-presenting protein according to claim 1, wherein one or plural amino acids are deleted, substituted, inserted, and/or added in the first to 130th amino acids sequence or the 140th to 238th amino acids sequence in the amino acid sequence of SEQ ID NO: 54, and exhibiting a fluorescence generation activity.

3. The peptide-presenting protein according to claim 1, wherein amino acid sequence(s) of end(s) of the inserted or substituted peptide consisting of four to fifteen amino acids are translated from base sequence(s) comprising a restriction enzyme site.

4. A fusion protein wherein the peptide-presenting protein according to claim 1, and a surface protein are fused.

5. A polynucleotide encoding the peptide-presenting protein according to claim 1.

6. A vector comprising the polynucleotide according to claim 5.

7. A host comprising the vector according to claim 6.

8. A library comprising the host according to claim 7, wherein the presented peptides are a number of peptides consisting of random amino acid sequences.

9. A protein for presenting a peptide, which can present the peptide in a presentation region consisting of the 131st to 139th amino acids in the amino acid sequence of the SEQ ID NO: 54, wherein the peptide consisting of four to fifteen amino acids is inserted in the 131st to 139th amino acids sequence, or substituted for any of 1 to 9 amino acid(s) sequence in the 131st to 139th amino acids sequence; and a total length of amino acids sequence corresponding to the 131st to 139th amino acids sequence is fifteen or less.

10. The protein for presenting a peptide according to claim 9, wherein one or plural amino acids are deleted, substituted, inserted, and/or added in the first to 130th amino acids sequence in the amino acid sequence or the 140th to 238th amino acids sequence in the amino acid sequence of SEQ ID NO: 54, and exhibiting a fluorescence generation activity.

11. The protein for presenting a peptide according to claim 9, wherein amino acid sequence(s) of end(s) of the inserted or substituted peptide consisting of four to fifteen amino acids are translated from base sequence(s) comprising a restriction enzyme site.

12. A fusion protein wherein the protein for presenting a peptide according to claim 9 is fused with a surface protein.

13. A polynucleotide encoding the protein for presenting a peptide according to claim 9.

14. A vector comprising the polynucleotide according to claim 13.

15. A host comprising the vector according to claim 14.

16. A method for constructing a library, comprising the steps of:
(1) inserting a nucleotide encoding a peptide having four to 15 amino acids into the vector according to claim 14 to obtain an expression vector, wherein the peptide is inserted in the 131st to 139th amino acids sequence of the presentation region, or substituted for any of 1 to 9 amino acid(s) sequence in the 131st to 139th amino acids sequence of the presentation region; and a total length of amino acids sequence corresponding to the 131st to 139th amino acids sequence is fifteen or less, and
(2) introducing the vector into the hosts.

17. A method for screening a peptide binding to a target molecule, comprising the steps of:
(1) bringing into contact the peptide library according to claim 8 with the target molecule,
(2) recovering hosts binding to the target molecule.

18. A method for screening a peptide according to claim 17, further comprising (3) the step of proliferating the recovered hosts.

19. The method for screening a peptide according to claim 18, wherein the contact step (1), the host-recovering step (2), and the proliferating step (3) are repeated.


SEQUENCE LISTING (77 sequences)

SEQ ID NO: 1 - DNA, 33 bp, Artificial
  Primer1 for enhanced GFP
tctagaggatccatggtgagcaagggcgaggag

SEQ ID NO: 2 - DNA, 60 bp, Artificial
  Primer2 for enhanced GFP
tagggcgaattcttaatggtgatggtgatgatggctgcccttgtacagctcgtccatgcc

SEQ ID NO: 3 - DNA, 60 bp, Artificial
  Primer 3 for mutation
cacaagttcagcgtgcgcggcgagggcgagggcgatgccaccaacggcaagctgaccctg

SEQ ID NO: 4 - DNA, 60 bp, Artificial
  Primer 4 for mutation
cagggtcagcttgccgttggtggcatcgccctcgccctcgccgcgcacgctgaacttgtg

SEQ ID NO: 5 - DNA, 51 bp, Artificial
  Primer 5 for mutation
caggagcgcaccatcagcttcaaggacgacggcacctacaagacccgcgcc

SEQ ID NO: 6 - DNA, 51 bp, Artificial
  Primer 6 for mutation
ggcgcgggtcttgtaggtgccgtcgtccttgaagctgatggtgcgctcctg

SEQ ID NO: 7 - DNA, 33 bp, Artificial
  Primer 7 for mutation
ctgagcacccagtccgtgctgagcaaagacccc

SEQ ID NO: 8 - DNA, 33 bp, Artificial
  Primer 8 for mutation
ggggtctttgctcagcacggactgggtgctcag

SEQ ID NO: 9 - DNA, 34 bp, Artificial
  Primer 9 for mutation
caagctggagtacaacttcaacagccacaacgtc

SEQ ID NO: 10 - DNA, 34 bp, Artificial
  Primer 10 for mutation
gacgttgtggctgttgaagttgtactccagcttg

SEQ ID NO: 11 - DNA, 33 bp, Artificial
  Primer 11 for mutation
cacaacgtctatatcaccgccgacaagcagaag

SEQ ID NO: 12 - DNA, 33 bp, Artificial
  Primer 12 for mutation
cttctgcttgtcggcggtgatatagacgttgtg

SEQ ID NO: 13 - DNA, 34 bp, Artificial
  Primer 13 for mutation
gaagaacggcatcaaggcgaacttcaagatccgc

SEQ ID NO: 14 - DNA, 34 bp, Artificial
  Primer 14 for mutation
gcggatcttgaagttcgccttgatgccgttcttc

SEQ ID NO: 15 - DNA, 34 bp, Artificial
  Primer 15 for mutation
caagatccgccacaacgtggaggacggcagcgtg

SEQ ID NO: 16 - DNA, 34 bp, Artificial
  Primer 16 for mutation
cacgctgccgtcctccacgttgtggcggatcttg

SEQ ID NO: 17 - DNA, 30 bp, Artificial
  Primer 17 (m1)
gatgaagttgatgagtacaacttcaacagc

SEQ ID NO: 18 - DNA, 18 bp, Artificial
  Primer 18 (m1)
caggatgttgccgtcctc

SEQ ID NO: 19 - DNA, 48 bp, Artificial
  Primer 19 (m2)
aacatcctggggcacaaggatgaggttgatttcaacagccacaacgtc

SEQ ID NO: 20 - DNA, 47 bp, Artificial
  Primer 20 (m2)
acgttgtggctgttgaaatcaacctcatccttgtgccccaggatgtt

SEQ ID NO: 21 - DNA, 30 bp, Artificial
  Primer 21 (m3)
gatgaagttgatgacaagcagaagaacggc

SEQ ID NO: 22 - DNA, 18 bp, Artificial
  Primer 22 (m3)
gacgttgtggctgttgaa

SEQ ID NO: 23 - DNA, 36 bp, Artificial
  Primer 23 (m4)
ggcggcgatgaagttgatgggcacaagctggagtac

SEQ ID NO: 24 - DNA, 18 bp, Artificial
  Primer 24 (m4)
cttgaagtcgatgccctt

SEQ ID NO: 25 - DNA, 36 bp, Artificial
  Primer 25 (m5)
gatgaagttgatggcagcgggcacaagctggagtac

SEQ ID NO: 26 - DNA, 18 bp, Artificial
  Primer 26 (m5)
cttgaagtcgatgccctt

SEQ ID NO: 27 - DNA, 18 bp, Artificial
  Primer 27 (m6)
gatgaagttgatggcatc

SEQ ID NO: 28 - DNA, 18 bp, Artificial
  Primer 28 (m6)
gaagtcgatgcccttcag

SEQ ID NO: 29 - DNA, 36 bp, Artificial
  Primer 29 (m7)
ggcgatgaagttgatggcatcctggggcacaagctg

SEQ ID NO: 30 - DNA, 18 bp, Artificial
  Primer 30 (m7)
gtcgatgcccttcagctc

SEQ ID NO: 31 - DNA, 30 bp, Artificial
  Primer 31 (m12)
gatgaagttgatgggcacaagctggagtac

SEQ ID NO: 32 - DNA, 18 bp, Artificial
  Primer 32 (m12)
cttgaagtcgatgccctt

SEQ ID NO: 33 - DNA, 18 bp, Artificial
  Primer 33 (m13)
cacaagctggagtacaac

SEQ ID NO: 34 - DNA, 27 bp, Artificial
  Primer 34 (m13)
atcaacttcatccttgaagtcgatgcc

SEQ ID NO: 35 - DNA, 18 bp, Artificial
  Primer 35 (m14)
aagctggagtacaacttc

SEQ ID NO: 36 - DNA, 27 bp, Artificial
  Primer 36 (m14)
atcaacttcatccttgaagtcgatgcc

SEQ ID NO: 37 - DNA, 33 bp, Artificial
  Primer 37 (m15)
ggcgatgaagttgatggcctggggcacaagctg

SEQ ID NO: 38 - DNA, 18 bp, Artificial
  Primer 38 (m15)
gaagtcgatgcccttcag

SEQ ID NO: 39 - DNA, 30 bp, Artificial; identical to SEQ ID NO: 31
  Primer 39 (m16)

SEQ ID NO: 40 - DNA, 18 bp, Artificial
  Primer 40 (m16)
gaagtcgatgcccttcag

SEQ ID NO: 41 - DNA, 33 bp, Artificial
  Primer 41 (m18)
tccgacgaggtggacgggcacaagctggagtac

SEQ ID NO: 42 - DNA, 21 bp, Artificial
  Primer 42 (m18)
gccgtcctccttgaagtcgat

SEQ ID NO: 43 - DNA, 36 bp, Artificial
  Primer 43 (m19)
tccgacgaggtggactccgggcacaagctggagtac

SEQ ID NO: 44 - DNA, 21 bp, Artificial
  Primer 44 (m19)
gccgtcctccttgaagtcgat

SEQ ID NO: 45 - DNA, 39 bp, Artificial
  Primer 45 (m20)
tccggggacgaggtggactccgggcacaagctggagtac

SEQ ID NO: 46 - DNA, 21 bp, Artificial
  Primer 46 (m20)
gccgtcctccttgaagtcgat

SEQ ID NO: 47 - DNA, 42 bp, Artificial
  Primer 47 (mH1)
ccggcgagtccggcggatcgagaagtggggcacaagctggag

SEQ ID NO: 48 - DNA, 16 bp, Artificial
  Primer 48 (mH1)
gccgtcctccttgaag

SEQ ID NO: 49 - DNA, 52 bp, Artificial
  Primer 49 (mH2)
cttcaaggaggacggctgcgatggtttttatgcgtgcgggcacaagctggag

SEQ ID NO: 50 - DNA, 52 bp, Artificial
  Primer 50 (mH2)
ctccagcttgtgcccgcacgcataaaaaccatcgcagccgtcctccttgaag

SEQ ID NO: 51 - DNA, 33 bp, Artificial
  Primer 51 (mH3)
aaatgctgctatagtctggggcacaagctggag

SEQ ID NO: 52 - DNA, 16 bp, Artificial
  Primer 52 (mH3)
gccgtcctccttgaag

SEQ ID NO: 53 - DNA, 717 bp, Artificial
  sfGFP seq
atgcgtaaaggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggcgaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactctcggcatggacgagctgtacaagtaa

SEQ ID NO: 54 - protein, 238 aa, Artificial
  sfGFP pep
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 55 - protein, 12 aa, Artificial
  mH1 peptide
KEDGKCCYSLGH

SEQ ID NO: 56 - protein, 13 aa, Artificial
  mH2 peptide
KEDGCDGFYACGH

SEQ ID NO: 57 - protein, 11 aa, Artificial
  mH3 peptide
KEDGFHAHPGH

SEQ ID NO: 58 - DNA, 45 bp, Artificial
  Primer 58 (m22)
gggtccggggacgaggtggacgggtccgggcacaagctggagtac

SEQ ID NO: 59 - DNA, 21 bp, Artificial
  Primer 59 (m22)
gccgtcctccttgaagtcgat

SEQ ID NO: 60 - DNA, 48 bp, Artificial
  Primer 60 (m8)
aacatcctggggcacgatgaggttgataacttcaacagccacaacgtc

SEQ ID NO: 61 - DNA, 48 bp, Artificial
  Primer 61 (m8)
gacgttgtggctgttgaagttatcaacctcatcgtgccccaggatgtt

SEQ ID NO: 62 - DNA, 18 bp, Artificial
  Primer 62 (m9)
gagtacaacttcaacagc

SEQ ID NO: 63 - DNA, 30 bp, Artificial
  Primer 63 (m9)
atcaacttcatccaggatgttgccgtcctc

SEQ ID NO: 64 - DNA, 21 bp, Artificial
  Primer 64 (m10)
gttgataagctggagtacaac

SEQ ID NO: 65 - DNA, 21 bp, Artificial
  Primer 65 (m10)
ttcatcgttgccgtcctcctt

SEQ ID NO: 66 - DNA, 21 bp, Artificial
  Primer 66 (m11)
gttgatcacaagctggagtac

SEQ ID NO: 67 - DNA, 21 bp, Artificial
  Primer 67 (m11)
ttcatcgccgtcctccttgaa

SEQ ID NO: 68 - DNA, 27 bp, Artificial
  Primer 68 (m17)
aagggcatcgacttcgatgaagttgat

SEQ ID NO: 69 - DNA, 27 bp, Artificial
  Primer 69 (m17)
atcaacttcatcgaagtcgatgccctt

SEQ ID NO: 70 - DNA, 30 bp, Artificial
  Primer 70 (m21)
ggggacgaggtggactccgggcacaagctg

SEQ ID NO: 71 - DNA, 30 bp, Artificial
  Primer 71 (m21)
cagcttgtgcccggagtccacctcgtcccc

SEQ ID NO: 72 - DNA, 36 bp, Artificial
  Primer 72 (mH4)
tggtacagctggctgctggggcacaagctggagtac

SEQ ID NO: 73 - DNA, 18 bp, Artificial
  Primer 73 (mH4)
gccgtcctccttgaagtc

SEQ ID NO: 74 - DNA, 36 bp, Artificial
  Primer 74 (mH5)
tggtacgcctggatgctggggcacaagctggagtac

SEQ ID NO: 75 - DNA, 18 bp, Artificial
  Primer 75 (mH5)
gccgtcctccttgaagtc

SEQ ID NO: 76 - protein, 12 aa, Artificial
  mH4 peptide
KEDGWYAWMLGH

SEQ ID NO: 77 - protein, 12 aa, Artificial
  mH5 peptide
KEDGWYSWLLGH